(19) United States
(12) Patent Application Publication
(10) Pub. No.: US 2025/0261834 A1
(43) Pub. Date: Aug. 21, 2025

(54) Methods and Devices Configured To Prevent Aspiration

(71) Applicant: Emory University, Atlanta, GA (US)

(72) Inventor: Anand Jain, Atlanta, GA (US)

(21) Appl. No.: 19/055,326

(22) Filed: Feb. 17, 2025

Related U.S. Application Data
(60) Provisional application No. 63/553,891, filed on Feb. 15, 2024.

(51) Int. Cl. A61B 1/00

(52) U.S. Cl. CPC A61B 1/00131; A61B 1/00128

(57) ABSTRACT

The present disclosure relates to systems and methods for preventing aspiration. In one implementation, the systems may include devices that can be used during gastrointestinal (GI) endoscopic procedures to prevent pulmonary aspiration and avoid esophageal pinch injury. The device may include a tube including a central lumen disposed along its length. The central lumen may be configured to receive an instrument. The device may include an occluder disposed at an end and including a lumen along its length. The occluder may have an outer surface that extends between a first circumference at its first end and a third circumference at its second end and a lumen that extends between the first circumference and the second circumference at its second end. The occluder may be configured to reversibly collapse to one or more collapsed configurations with respect to the tube. The occluder may be biased to an expanded configuration.

### CROSS-REFERENCE TO RELATED APPLICATIONS

This application claims the benefit of U.S. Provisional Application No. 63/553,891 filed Feb. 15, 2024. The entirety of this application is hereby incorporated by reference for all purposes.

### BACKGROUND

Upper gastrointestinal (GI) endoscopic procedures are commonly performed to investigate, diagnose, and/or treat conditions affecting the upper part of the digestive system (esophagus, stomach, duodenum). During these procedures, an endoscope is threaded through the mouth and into the digestive system so that a clinician can diagnose and/or treat conditions of the upper part of the digestive system, such as the esophagus, the stomach, and an upper portion of the small intestine (e.g., duodenum). Normally, this procedure is performed and/or attempted to be performed under sedation, for example, using propofol, without general anesthesia. However, this procedure can have a risk of pulmonary aspiration from retained stomach or esophageal continents. This risk can be increased for those patients who have certain conditions (e.g., patients with gastroesophageal reflux disease who have an incompetent lower esophageal sphincter barrier).

To reduce this risk, endoscopic GI procedures will generally be performed under general anesthesia; thereby increasing the risk of complications associated with the procedure, as well as resource and financial costs. Additionally, during GI endoscopic procedures, patients can be at risk of pharyngeal perforation as a result of a pinch injury. This can occur when the esophageal diameter is larger than the diameter of the endoscope but smaller than a typical overtube. As the overtube advances over the endoscope, pharyngeal or esophageal tissue can involute in between the endoscope and the overtube, and a certain amount of force exerted on the pinched pharyngeal or esophageal tissue can cause it to perforate.

### SUMMARY

Thus, there is a need for an overtube that can successfully reduce the potential for GI fluid to be aspirated into the lungs while allowing for an endoscopic procedure to be performed under sedation without general anesthesia and preventing pharyngeal or esophageal pinch injury.

The devices disclosed herein relates generally to endoscopic overtubes that can be used during GI endoscopic procedures to prevent pulmonary aspiration and avoid esophageal pinch injury.

In some examples, the devices may include a device that includes a tube. The tube may have a first end, a second end, and a length there between. The tube may include a central lumen. The device may also include an occluder disposed at the second end of the tube. The occluder may have a first end, a second end, and a length there between. The occluder may include a lumen disposed along the length. The occluder may have a first circumference at the first end, and a second circumference and a third circumference at the second end. The third circumference may be larger than the first circumference and the second circumference. The occluder may have an outer surface extending between the first circumference and the third circumference. In some examples, the occlude may include a lumen extending between the first circumference and the second circumference. The occluder may be configured to reversibly collapse to one or more collapsed configurations with respect to the tube. The occluder may be biased to an expanded configuration.

In some examples, the device may also include an adapter. The adapter may be disposed at the first end of the tube. In some examples, the adapter may include a clip configured to attached onto an instrument. In some examples, the second circumference may correspond to the central lumen and/or surround the tube.

In some examples, the methods may include a method of occluding a lumen of a body. The method may include providing the device. The method may include advancing the device over an instrument disposed within a body lumen to a region of interest within the body lumen so that the occluder engages surrounding tissue of the body lumen while the device is advanced until the device reaches the region of interest. In some examples, if the device encounters resistance from the surrounding tissue when advanced along the body lumen towards the region of interest, the occluder may move from the expanded configuration to the one or more of the collapsed configurations. In some examples, when the device is at the region of interest, the occluder may be in a more expanded configuration so as to occlude the body lumen. The method may include performing a procedure with the other device when the occluder is in the more expanded configuration at the region of interest.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows an isometric view of a device according to embodiments.

FIG. 2 shows a top view of the device shown in FIG. 1.

FIG. 3 shows a bottom view of the device shown in FIG. 1.

FIG. 4 shows a side view of the device shown in FIG. 1.

FIG. 5A shows a front view of the device shown in FIG. 1.

FIG. 5B shows a cross-sectional view of the device as shown in FIG. 5A.

FIG. 6 shows a cross-sectional view of the device shown in FIG. 1 according to embodiments.

FIG. 7 shows another cross-sectional view of the device shown in FIG. 1 according to embodiments.

FIG. 8 shows an example of a prototype of the device according to embodiments.

FIGS. 9A and 9B show views of the device shown in FIGS. 1-7 in an example of a collapsed configuration according to embodiments. FIG. 9A shows an isometric view of the device in the example of the collapsed configuration and FIG. 9B shows a bottom view of the device shown in FIG. 9A.

FIGS. 10A and 10B show views of the device shown in FIGS. 1-7 in another example of a collapsed configuration according to embodiments. FIG. 10A shows an isometric view of the device in the other example of the collapsed configuration and FIG. 10B shows a bottom view of the device shown in FIG. 10A.

FIGS. 11A and 11B show views of the device shown in FIGS. 1-7 in another example of a collapsed configuration according to embodiments. FIG. 11A shows an isometric view of the device in the other example of the collapsed configuration and FIG. 11B shows a bottom view of the device shown in FIG. 11A.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments may relate to devices that can be used as an endoscopic overtube that can prevent pulmonary aspiration and avoid esophageal pinch injury, such as, during GI endoscopic procedures. In some embodiments, the device may include an occluder configured to occlude a body lumen in which it is delivered to prevent the outflow of contents, such as those in the stomach. In some examples, when in its expanded configuration, the occluder may be configured to splay the tissue of the body lumen so as to minimize or prevent a pinch injury in that tissue. By way of example, the occluder may be configured to splay the pharyngeal or esophageal tissue during the advancement of the device into the esophagus, such as to minimize the risk of pharyngeal or esophageal pinch injury. In some examples, the occluder may include spaced internal ribs configured to stabilize the occluder and to bias the occluder to its expanded configuration as it is passed through a body lumen, such as the esophagus. In some examples, the occluder may have a diameter wider than the body/tube of the device so that it can be configured to displace/splay the esophageal tissue at the leading edge as the device is advanced, creating a path for the device and minimizing risk of pinch injury. This way, the occluder may have three functions: 1) block refluxate, 2) stabilize position of the tube; and 3) splay the pharyngeal and esophageal tissue during advancement of the device so as to minimize the risk of crimp/pinch injury.

The devices and methods of the disclosure are described with respect to upper gastrointestinal procedures and esophagus. For example, the disclosed devices and methods can be used with another instrument, such as any gastrointestinal endoscope, and can be configured to prevent aspiration and pinch injury so that a diagnostic or investigational procedure may be performed in the gastrointestinal region without the need of general anesthesia. However, it will be understood that the devices and methods are not limited to this type of gastrointestinal procedure and device. The devices and methods may be used with an endoscope for any gastrointestinal procedure performed within the esophagus or a different body lumen, with an endoscope for a procedure performed in a different body lumen, with another instrument for a different procedure performed in a different body lumen, by itself, or any combination thereof. For example, the devices and methods can be used with a surgical laparoscope, colonoscope, small bowel enteroscope, ureteroscope, hysteroscope, among others, or any combination thereof. By way of another example, the devices and methods may be used with any procedure to improve visualization by displacing areas of tissue within a body lumen to provide a clear path. For example, the devices and methods may be used with an endoscope during a pancreatic necrosectectomy to improve visualization.

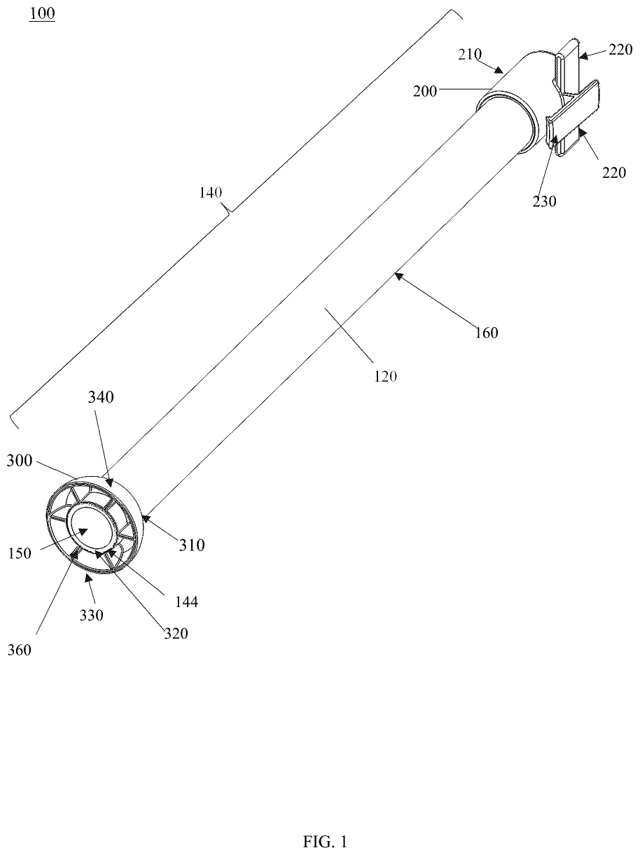

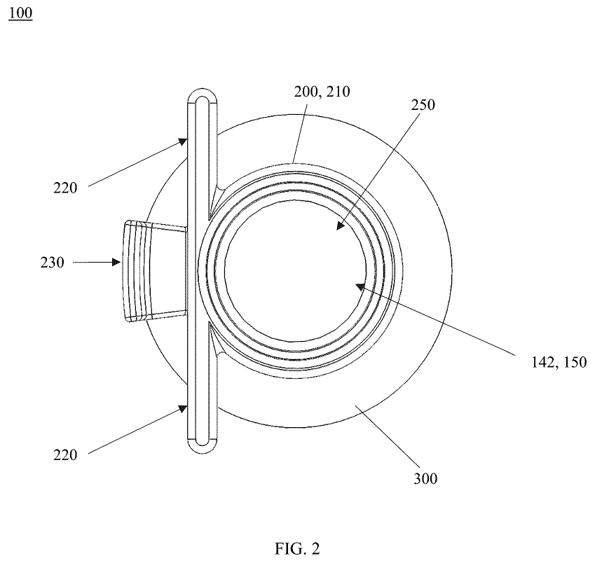

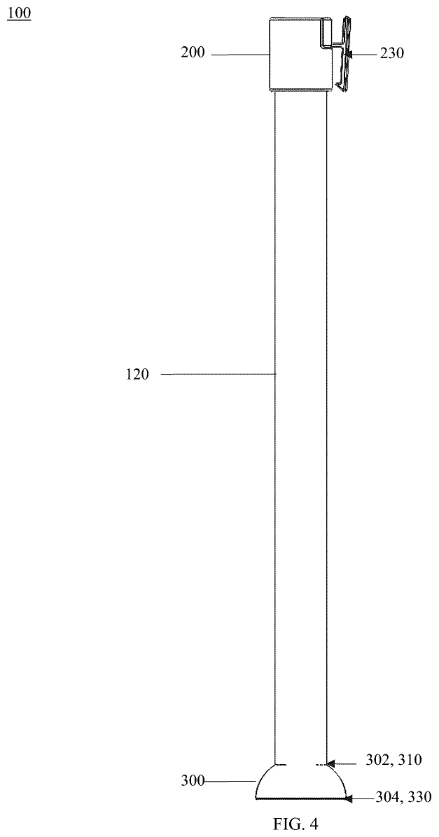

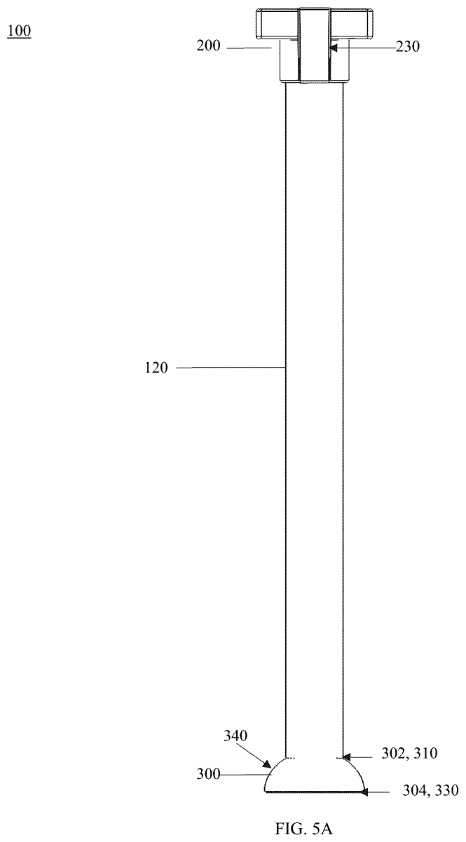

FIGS. 1-11B show an example of device 100 according to some embodiments. FIGS. 1-11B shows an example of the device 100 in which the device 100 can act as a flexible overtube. In some embodiments, the device 100 may include a body or tube 120. In some embodiments, the tube 120 may be configured to surround the sheath or tube of another instrument, such as a GI endoscope.

In some embodiments, the tube 120 may have a first end 142, a second end 144, and a length 140 there between. In some embodiments, the tube 120 may have a central lumen 150 disposed along the length 140. The central lumen 150 may be configured to receive and surround an insertable portion of an instrument, such as a GI endoscope. In some embodiments, the diameter of the central lumen 150 may be sized so that the instrument, such as a gastrointestinal instrument, such as GI endoscope, can advance/retract through to/from the desired procedural region.

In some embodiments, the tube 120 may be made of one or more biocompatible materials. For example, the one or more biocompatible materials may be flexible. By way of example, the one or more materials may include but is not limited to polybutylene succinate (PBS), polylactide (PLA), acrylate butadiene styrene (ABS), resin, silicone, among others, or in a combination thereof.

In some embodiments, the tube 120 may be transparent. In some examples, the tube 120 may be semi-transparent or fully transparent.

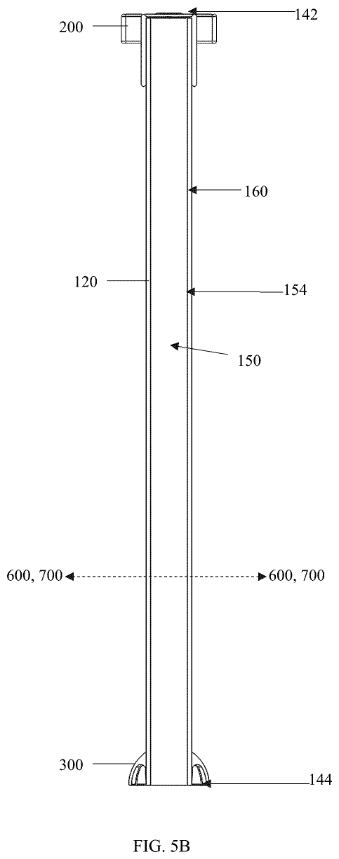

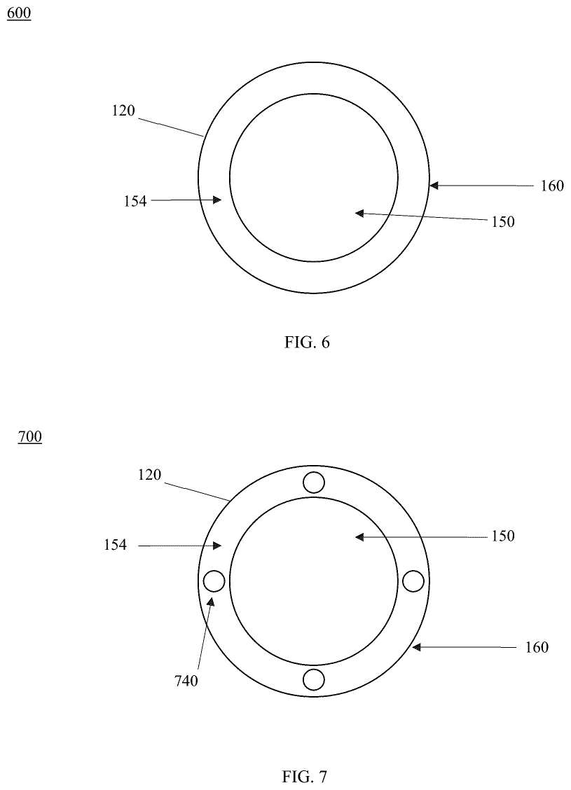

In some embodiments, the tube 120 may have an outer surface 160. In some embodiments, the tube 120 may include a wall 154 disposed between the central lumen 150 and the outer surface 160. In some examples, as shown in the cross-section 600 in FIG. 6, the wall 154 of the tube 120 may be solid. In some examples, as shown in the cross-section 700 shown in FIG. 7, the wall 154 of the tube 120 may include one or more channels 740 configured to receive and/or deliver one or more instruments, such as probe(s), suction instrument(s), irrigation instrument(s), among other instrument(s), or any combination thereof.

In some embodiments, the dimensions (e.g., length, diameter, etc.) and/or other features (i.e., flexibility/stiffness) of the tube 120 may depend on the delivery site and/or instruments to be delivered. For example, the tube 120 may be stiffer, longer, and wider when used as a laparoscopic surgical port where instruments need to be advanced deep into the abdomen.

In some embodiments, the device 100 may include one or more features (not shown) disposed on the outer surface 160 of the tube 120. By way of example, the one or more additional features may include but is not limited to one or more of: inflatable balloons, stents, stitching devices, snares, ablation devices, hooks for loop reduction, sensors, medications, biocompatible lubricants, among others, or any combination thereof.

In some embodiments, the device 100 may include an adapter 200 disposed at the first end 142 and an occluder 300 disposed at the second end 144. In some embodiments, the adapter 200 and the occluder 300 may partially surround the tube 120 and/or have an opening that corresponds to the central lumen 150.

In some examples, the adapter 200 may be disposed on the outer surface 160 of the tube 120 and may have an opening 250 that corresponds to the central lumen 150. In some embodiments, the adapter 200 may have an anchoring member 230 configured to anchor to another instrument or device, such as a bite guard. For example, the anchoring member 230 may be configured to clip onto a bite guard. In some examples, the anchoring member 230 may include a set of wings 220 disposed on opposing sides.

In some embodiments, the adapter 200 may be made of one or more biocompatible materials. By way of example, the one or more materials may include but is not limited to polybutylene succinate (PBS), polylactide (PLA), acrylate butadiene styrene (ABS), resin, silicone, among others, or in a combination thereof.

In some examples, the device 100 may have an alternative adapter or no adapter. For example, the geometry and shape of the adapter 200 may depend on the site of the procedure and/or instrument or device to which to be anchored. By way of example, the adapter 200 may have a different-shaped anchoring member. For example, the adapter 200 may have a different anchoring member 230 configured to anchor to an instrument or device.

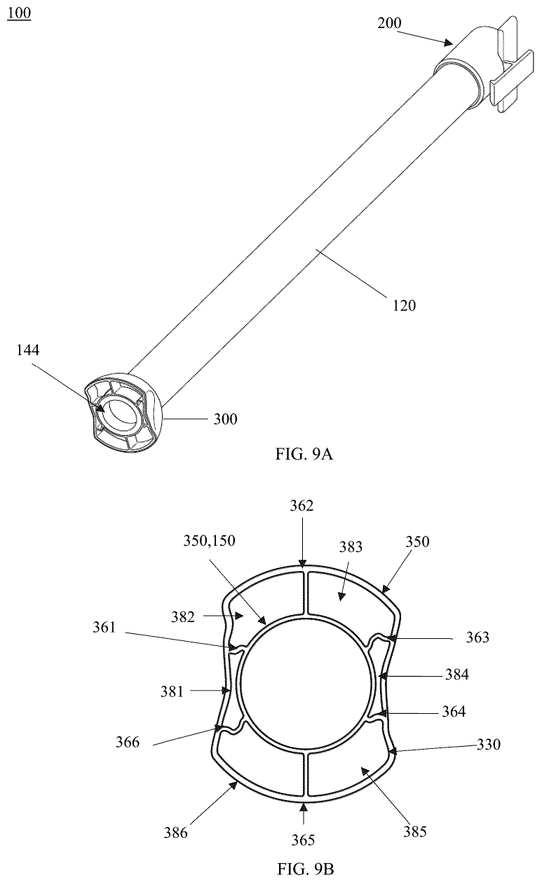

In some embodiments, the occluder 300 may have a first end 302, a second end 304, and a length there between. In some examples, the occluder 300 may be configured to move between an expanded configuration with respect to the tube 120 shown in FIGS. 1-7 and one or more collapsed configurations with respect to the tube 120, for example, as shown in FIGS. 9A-11B. In some examples, the occluder 300 shown in FIGS. 1-7 may be biased to the expanded position as shown in FIGS. 1-7 so that the expanded configuration is the default configuration. In this example, the occluder 300 may be configured to collapse from the expanded configuration to a collapsed configuration upon application of force and/or resistance, for example, by surrounding body lumen. For example, one or more sections of the occluder 300 may be configured to reversibly collapse with respect to the tube 120 when advanced through a body lumen having a circumference that is smaller than the occluder 300. This way, the occluder 300 may easily and reversibly conform to the patient's anatomy by enabling displacement of the surrounding tissue laterally and distally, such as to allow smooth advancement of the device 100 and minimize tissue trauma. FIGS. 9A-11B show examples of the device 100 in collapsed configurations.

In some embodiments, the occluder 300 may have a first circumference 310 disposed at the first end 302, and a second circumference 320 and a third circumference 330 disposed at the second end 304. The third circumference 330 may correspond to an outer circumference and the second circumference 320 may correspond to an inner circumference at the second end 304. In some examples, the third circumference 330 may be larger than the first circumference 310 and the second circumference 320. In some embodiments, the occluder 300 may include a central lumen 350 that extends between the first circumference 310 and the second circumference 320. The central lumen 350 may correspond to the inner surface of the occluder 300. In some examples, the occluder 330 may include at least one opening 352 disposed at the second end 304 that interfaces with the central lumen 350.

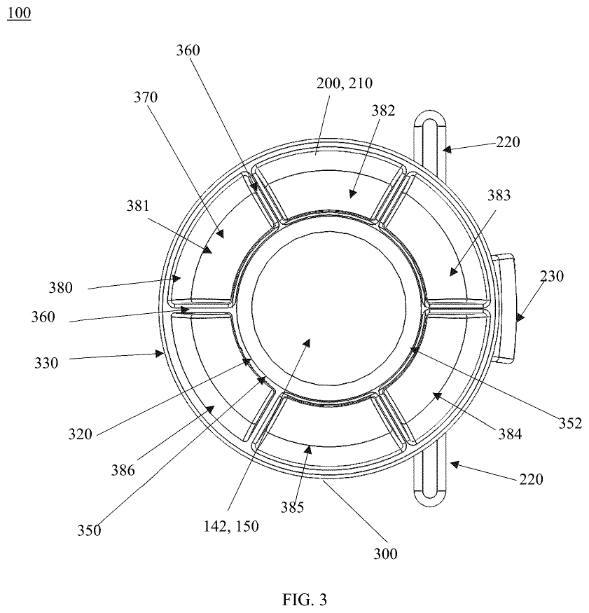

In some examples, the occluder 300 may at least partially surround the outer surface 160 of the tube 120. For example, at least a portion of the tube 120 may be disposed within the central lumen 350, for example, as shown in FIGS. 3-5B. In some examples, the second end 304 of the occluder 300 may align with the second end 144 of the tube 120 so that the opening 352 corresponds to and interfaces with the central lumen 150. In other examples, the occluder 300 may be integrated with the tube 120.

In some embodiments, the occluder 300 may have an outer surface 340 extending between the first circumference 310 and the third circumference 330. In some examples, the outer surface 340 of the occluder 300 may flair (taper) between the first circumference 310 to the third circumference 330 when in the expanded configuration.

In some embodiments, the occluder 300 may include a plurality of ribs 360 spaced disposed so as to extend along the length between the second circumference 320 and the third circumference 330. In some embodiments, the plurality of ribs 360 may be spaced apart with respect to the central lumen 350 by an empty space 370 between each set of ribs 360 and the third circumference 330 and the second circumference 320. In some embodiments, the plurality of ribs 360 may be evenly spaced with respect to the second circumference 320 and the third circumference 330 of the occluder 300. In some embodiments, the dimensions of the plurality of the ribs 360 may be substantially the same. The plurality of ribs 360 may include any number of ribs and is not limited to the six ribs (the 361, 362, 363, 364, 365, and 366) shown.

In some embodiments, the occluder 300 may include a plurality of sections 380. In some examples, each section 380 may correspond to the empty space 370 defined by a set of ribs 360, the second circumference 320, and the third circumference 330. In some examples, the sections 380 may have substantially the same dimensions, as shown. In other examples, the sections 380 may have different dimensions.

In some embodiments, the occluder 300 may include six sections 380 as shown in FIGS. 1-7 and 9A-11B. For example, the sections 380 may include a first section 381 defined by the set of ribs 361 and 366; a second section 382 defined by the set of ribs 361 and 362; a third section 383 defined by the set of ribs 362 and 363; a fourth section 384 defined by the set of ribs 363 and 364; a fifth section 385 defined by a set of ribs 364 and 365; and a six section 386 defined by a set of ribs 365 and 366. In some embodiments, the occluder 300 may include more or less sections 380.

When in the default, expanded configuration, for example, as shown in FIGS. 1-7, the ribs 360 of the occluder 300 may be elongated so that they extend between the second circumference 320 and the third circumference 330. In this configuration, the outer surface 340 may be separated by the inner surface/central lumen 350 by the ribs 360 so that the outer surface 340 flairs between the first circumference 310 to the third circumference 330. For example, as shown in FIGS. 1-7, when the occluder 300 is in an expanded configuration, the ribs 360 may be substantially straight between the second circumference 320 and the third circumference 330.

When in a collapsed configuration, at least a portion of the outer surface 340 of the occluder 300 may be pressed towards or against the inner surface/central lumen 350. In the collapsed configuration, one or more ribs 360 may be bent between the second circumference 320/outer surface 340 and the third circumference 330/central lumen 350 causing the respective section(s) including the one or more bent rib(s) 360 to at least partially collapse. When one or more ribs 360 of a section bends, it may cause at least a portion of the outer surface 340 of the respective section(s) 380 to move towards the inner surface/central lumen 350 causing that section(s) to at least partially collapse. This can result in at least the outer surface 340 of the occluder to change shape. In some examples, depending on the outside force, a portion of the inner surface/central lumen 350 and the corresponding section 380 and the corresponding portion of the tube 120 may also collapse causing the occluder 300, including the inner surface/central lumen 350, and corresponding portion of the tube 120 to change shape.

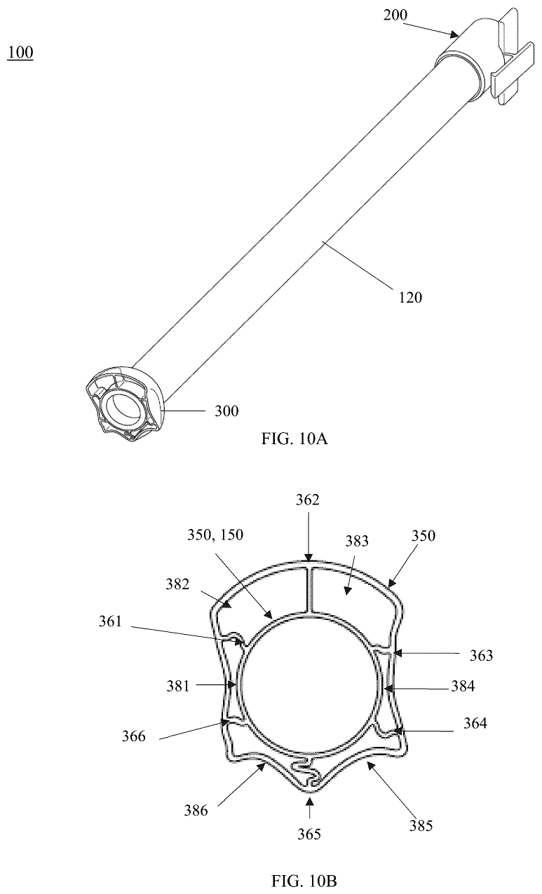

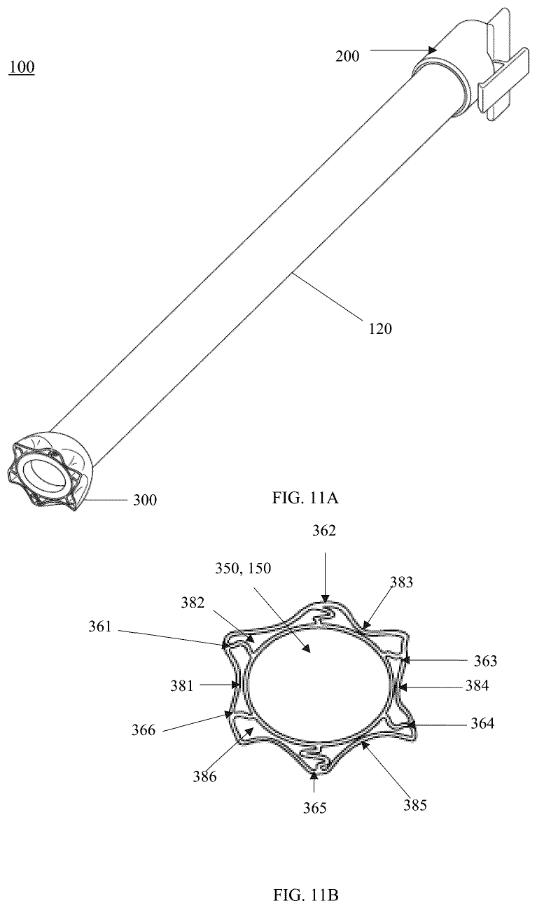

FIGS. 9A-11B show examples of the device 100 with the occluder 300 shown in FIGS. 1-7 in different collapsed configurations. FIGS. 9A-10B show examples of the occluder 300 in partially collapsed configurations and FIGS. 11A and B show examples of the occluder in a mostly collapsed configuration. These examples are nonlimiting and the occluder 300 can be configured to be in a different collapsed configuration depending on the resistance during delivery.

FIGS. 9A and B show an example of the occluder 300 in a collapsed configuration. In this example, the ribs 361, 363, 364, and 366 are bent resulting in at least partial collapse of all sections 381, 382, 384, 385, and 386 and a change in shape of the occluder 300 from the change in shape of the outer surface 340/third circumference 330. In some examples, the state of collapse of the corresponding section 380 may depend on whether one or both ribs of that section are bent. By way of example, as shown, the sections 381 and 384 for which both corresponding ribs (ribs 361 and 366 for the first section 381; and ribs 363 and 364 for the fourth section 384) are bent can be more collapsed than the other sections 381, 385, and 386.

FIGS. 10A and B show another example of the occluder 300 in a collapsed configuration. In this example, more of the ribs 360 are bent so that more sections are mostly collapsed. By way of example, the ribs 361, 363, 364, 365, and 366 are bent resulting in at least partial collapse of all sections 381-386 and a change in shape of the occluder 300 from the change in shape of the outer surface 340/third circumference 330. As shown, the sections 381, 384, 385, and 386 for which both corresponding ribs are bent can be more collapsed than the other sections 382 and 383.

FIGS. 11A and B show another example of the occluder 300 in a collapsed configuration. In this example, all of the ribs 360 are bent so that all sections are mostly collapsed. By way of example, all of the ribs 361-366 are bent resulting in the mostly collapse of all sections 380 and a change in shape of the occluder 300 from the change in shape of the outer surface 340/third circumference 330. As shown, the sections 381-386 for which both corresponding ribs are mostly collapsed. In this example, the mostly collapse of one or more sections 360 can also result in the change of shape of the inner surface/central lumen 350 and the tube 120 at the end 144.

In some embodiments, the occluder 300 may be made of one or more biocompatible materials. In some examples, the occluder 300 may be semi-flexible or flexible.

In some embodiments, the one or more biocompatible materials may have flexible properties. By way of example, the one or more materials may include but is not limited to polybutylene succinate (PBS), polylactide (PLA), acrylate butadiene styrene (ABS), resin, silicone, among others, or in a combination thereof.

In some examples, the outer surface 340 of the occluder 300 may have a different hardness/stiffness than the central lumen 350 of the occluder 300. For example, the outer surface 340 may be more flexible than the wall/inner surface of the central lumen 350 of the occluder 300.

In some examples, the occluder 300 may be more flexible than the tube 120. By way of example the occluder 300 may have a hardness of about 10-20 A; and the tube 120 may have a stiffness of about 40-60 A. In some examples, the occluder 300 may be about 10 A and the tube may be about 50 A.

In some embodiments, the occluder 300 may be transparent. In some examples, the occluder 300 may be semi-transparent or transparent.

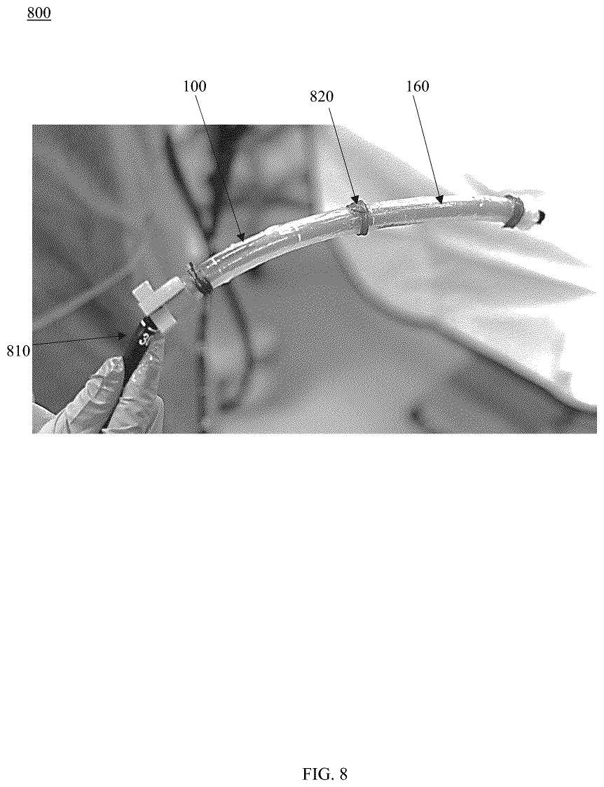

In some examples, as shown in an example 800 shown in FIG. 8, the device 100 may be disposed on an endoscope tube 810 before the advancement into a patient's esophagus. In some embodiments, the device 100 may include one or more sets of imaging markers 820 disposed on the outer surface 160 of the tube 120. For example, the one or more sets of imaging markers 820 can be set at a number of different distances, including but not limited to 5 cm, 10 cm, 15 cm, 20 cm, and/or 25 cm.

In some examples, the disclosed devices may be used in a procedure to redirect/block retrograde contents whilst maintaining an open central lumen to facilitate passage of an instrument, such as an endoscope or other instrument. For example, for a gastrointestinal procedure, after the patient is prepared for the procedure by providing access to the esophagus, such as inserting a mouthguard with a bit block opening the airway, an instrument, such as endoscope may be positioned to the desired location. After which, the disclosed device 100 may be inserted into a mouth of a patient advanced over an instrument, such as an endoscope, through the esophagus in anterograde motion until positioned at a desired location and the anchoring member 230 of the adapter 200 interfaces with the bite block clip provided at the patient's mouth. When initially inserted into the pharynx, the occluder 300 may be in one or more collapsed configurations due to the resistance. For example, any resistance during the device's advancement may cause one or more of the ribs 360 to bend so that one or more of the sections 380 collapses toward the central lumen 350 resulting in one or more of the collapsed configurations. During advancement into the esophagus, the occluder 300 of the device 100 may move to the expanded configuration or a less collapsed configuration (i.e., a more expanded configuration) so that the outer surface 340 engages the surrounding tissue. By way of example, the device 100 may move from a collapsed configuration shown in FIGS. 11A and B to a less collapsed/more expanded configuration shown in FIGS. 10A and B or FIGS. 9A and B or the expanded configuration shown in FIGS. 1-7.

The stiffness profile, shape, and structure of the occluder 300 can enable displacement of the surrounding tissue laterally and distally such as to allow smooth advancement of the device 100 and minimize tissue trauma. After the device 100 is advanced to the desired position and the anchoring member 230 interfaces with the complimentary bite block of the mouth guard, the instrument, such the endoscope or other instrument, can be advanced freely further into the esophagus to the site to conduct the procedure. After the procedure is completed, the disclosed device 100 can be removed whilst the endoscope is still in place or after endoscope has been removed.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and the appended claims.

What is claimed is:

1. A device, comprising:
a tube having a first end, a second end, and a length there between;
the tube including a central lumen disposed along the length, the central lumen configured to receive an instrument; and
an occluder disposed at the second end;
the occluder having a first end, a second end, and a length there between;
the occluder including a lumen along the length;
the occluder having a first circumference at the first end;
the occluder having a second circumference and a third circumference at the second end;
the third circumference being larger than the first circumference and the second circumference;
the occluder having an outer surface extending between the first circumference and the third circumference; and a lumen extending between the first circumference and the second circumference;
the occluder being configured to reversibly collapse to one or more collapsed configurations with respect to the tube; and
the occluder being biased to an expanded configuration.

2. The device according to claim 1, further comprising:
a plurality of ribs disposed along the length of the occluder;
wherein each rib extends between the second circumference and the third circumference; and
a plurality of sections, each section being defined by (i) a set of ribs of the plurality of ribs and (ii) the second circumference and the third circumference.

3. The device according to claim 2, wherein:
the plurality of ribs is spaced apart with respect to the lumen by an empty space; and
each empty space corresponding to a section.

4. The device according to claim 3, wherein:
in the expanded configuration, the plurality of ribs are elongated between the second circumference and the third circumference; and
in the one or more collapsed configurations, one or more of the plurality of the ribs are bent between the second circumference and the third circumference so that one or more sections defined by the one or more of the plurality of the ribs collapse towards the lumen of the occluder;
in the one or more collapsed configurations, the outer surface disposed in that section is pushed towards the lumen of the occluder.

5. The device according to claim 4, wherein:
the tube is made of one or more materials and the occluder is made of one or more materials; and
the one or more materials of the occluder is more flexible than the one or more materials of the tube.

6. The device according to claim 1, further comprising:
an adapter disposed at the first end.

7. The device according to claim 6, wherein:
the adapter is disposed on an outer surface of the tube; and
the adapter includes an anchor member configured to engage another device.

8. The device according to claim 6, wherein the other device is a mouthguard.

9. The device according to claim 1, wherein:
one or more surface features is disposed on the outer surface of the tube.

10. The device according to claim 9, wherein:
the one or more surface features includes one or more of inflatable balloons, stents, stitching devices, snares, ablation devices, hooks for loop reduction, sensors, medications, and/or biocompatible lubricants.

11. The device according to claim 1, wherein:
the tube includes an outer surface; and
the tube includes a wall between the lumen and the outer surface, the wall including one or more additional internal channels configured for one or more instruments.

12. The device according to claim 11, wherein the one or more instruments includes one or more probes, a suction instrument, an irrigation instrument, and/or another instrument.

13. The device according to claim 1, wherein the central lumen is configured to receive an endoscope.

14. A method of occluding a lumen of a body, comprising:
providing a device including:
a tube having a first end, a second end, and a length there between;
the tube including a central lumen disposed along the length, the central lumen configured to receive an instrument; and
an occluder disposed at the second end;
the occluder having a first end, a second end, and a length there between;
the occluder including a lumen along the length;
the occluder having a first circumference at the first end;
the occluder having a second circumference and a third circumference at the second end;
the third circumference being larger than the first circumference and the second circumference;
the occluder having an outer surface extending between the first circumference and the third circumference; and a lumen extending between the first circumference and the second circumference; and
the occluder being configured to reversibly collapse to one or more collapse configurations with respect to the tube; and
the occluder being biased to an expanded configuration;
advancing the device over an instrument disposed within a body lumen to a region of interest within the body lumen so that the occluder engages surrounding tissue of the body lumen while the device is advanced until it reaches the region of interest;
wherein if the device encounters resistance from the surrounding tissue when advanced along the body lumen towards the region of interest, the occluder moves from the expanded configuration to the one or more of the collapsed configurations; and
wherein when the device is at the region of interest, the occluder is in a more expanded configuration so as to occlude the body lumen; and
performing a procedure with the other device when the occluder is in the more expanded configuration at the region of interest.

15. The method according to claim 14, wherein the device further includes:
a plurality of ribs disposed along the length of the occluder;
wherein each rib extends between the second circumference and the third circumference; and
a plurality of sections, each section being defined by (i) a set of ribs of the plurality of ribs and (ii) the second circumference and the third circumference.

16. The method according to claim 15, wherein the device further includes:
the plurality of ribs is spaced apart with respect to the lumen by an empty space; and
each empty space corresponding to a section.

17. The method according to claim 16, wherein:
in the expanded configuration, the plurality of ribs are elongated between the second circumference and the third circumference; and
in the one or more collapsed configurations, one or more of the plurality of ribs are bent between the second circumference and the third circumference so that one or more sections defined by the one or more of the plurality of the ribs collapse towards the lumen of the occluder;
in the one or more collapsed configurations, the outer surface disposed in that section is pushed towards the lumen of the occluder.

18. The method according to claim 16, wherein:
the tube is made of one or more materials and the occluder is made of one or more materials; and
the one or more materials of the occluder is more flexible than the one or more materials of the tube.

19. The method according to claim 16, further comprising:
an adapter disposed at the first end;
wherein the adapter is disposed on an outer surface of the tube; and
wherein the adapter includes an anchor member configured to engage another device.

20. The method according to claim 19, wherein:
the other device is a mouthguard;
the instrument is an endoscope; and
the body lumen is an esophagus.
